(19) United States
(12) Patent Application Publication
(10) Pub. No.: US 2025/0234911 A1
(43) Pub. Date: Jul. 24, 2025

(54) NOVEL PHARMACEUTICAL OR NUTRACEUTICAL COMPOSITION FOR TREATING OR PREVENTING EPILEPSY

(71) Applicant: Zenvision Pharma LLP, Mumbai (IN)

(72) Inventors: Bobba Venkata Sivakumar, Navi Mumbai (IN); Sunil Pophale, Navi Mumbai (IN); Amit Manmode, Navi Mumbai (IN); Shankar Pol, Navi Mumbai (IN); Rupesh Kumar Gupta, Navi Mumbai (IN); Anoop Kumar Singh, Navi Mumbai (IN); Alok Kumar Singh, Navi Mumbai (IN); Piyush Kumar Singh, Navi Mumbai (IN)

(21) Appl. No.: 18/839,289

(22) PCT Filed: Feb. 17, 2023
(86) PCT No.: PCT/IN2023/050161

§ 371 Date: Aug. 16, 2024

(30) Foreign Application Priority Data
Feb. 17, 2022 (IN) 202121037238
Mar. 17, 2022 (IN) 202121042173

(51) Int. Cl. A23L 33/12; A23L 29/00; A23L 33/15; A23L 33/16; A23L 33/175

(52) U.S. Cl. CPC A23L 33/12; A23L 29/06; A23L 33/15; A23L 33/16; A23L 33/175

(57) ABSTRACT

The present invention relates to a novel pharmaceutical or nutraceutical composition comprising multivitamins, minerals, coenzyme, amino acids and omega-3 fatty acids, and process of preparation and its prophylactic or supportive use as an adjuvant to anticonvulsant medicines used for treating/preventing of various types of epilepsy or seizures.

### FIELD OF THE INVENTION

The present invention relates to a novel pharmaceutical or nutraceutical composition, comprising multivitamins, minerals, coenzyme, amino acids and omega-3 fatty acids, its process of preparation and prophylactic or supportive use of said pharmaceutical composition.

More particularly the present invention relates to a novel pharmaceutical or nutraceutical composition for oral administration comprising multivitamins, minerals, coenzyme, amino acids and omega-3 fatty acids; its process of preparation and its prophylactic or supportive use as an adjuvant to anticonvulsant medicines used for treating/preventing of various types of epilepsy or seizures.

More particularly the present invention relates to a novel pharmaceutical or nutraceutical composition for oral administration comprising multivitamins, minerals, coenzyme, amino acids and omega-3 fatty acids optimized for all age groups from paediatrics, adolescents, adults to elderly, its process of preparation and its prophylactic or supportive use as an adjuvant to anticonvulsant medicines used for treating/preventing of various types of epilepsy or seizures.

### BACKGROUND OF THE INVENTION

Epilepsy is a central nervous system (neurological) disorder in which brain activity becomes abnormal, causing seizures or periods of unusual behavior, sensations, and sometimes loss of awareness. Epilepsy affects both males and females of all races, ethnic backgrounds and ages.

Epilepsy affects around 50 million people worldwide. It is characterized by recurrent seizures, which are brief episodes of involuntary movement that may involve a part of the body (partial) or the entire body (generalized) and are sometimes accompanied by loss of consciousness and control of bowel or bladder function.

Seizure episodes are a result of excessive electrical discharges in a group of brain cells. Different parts of the brain can be the site of such discharges. Seizures can vary from the briefest lapses of attention or muscle jerks to severe and prolonged convulsions. Seizures can also vary in frequency, from less than 1 per year to several per day.

Types of epilepsy are as below:
1) Generalized seizures: “Grand mal” or generalized tonic-clonic, Absence seizures, Myoclonic seizures, Clonic seizures, Tonic seizures and Atonic seizures; and
2) Partial (focal) Seizure: Simple partial (no loss of awareness), Complex partial (impaired awareness) and Partial with secondary generalization

Anticonvulsants (antiepileptic drugs or antiseizure drugs) are a diverse group of pharmacological agents used in the treatment of epileptic seizures. Different types of drugs are used in the treatment of epilepsy according to seizures/epilepsy's category or type.

Although said anticonvulsant medications are found to be extremely helpful, these medications may not work for everyone, and as with any medication, can come with a risk of side effects.

There has been continuous research going on around the world for the development of alternative system of medicines or nutritional supplements, said medicines or nutritional supplements when used in combination with existing anticonvulsant medication may enhance antiepileptic action with minimal side effects.

Studies have illustrated that important physiological roles played by various vitamins, minerals, amino acids in epilepsy and established a correlation between deficiencies or excesses of these vitamins, minerals, amino acids and the etiologies of certain disease states like epilepsy in humans.

Hence, there is still exists a need to develop improved pharmaceutical or nutraceutical composition to overcome disadvantages associated with the products available in the market.

The inventors of the present invention surprisingly found that the use of synergistic combination of multivitamins, minerals, coenzyme, amino acids and omega-3 fatty acids which supplies the right amount of the right vitamins, minerals, coenzymes, amino acids and omega-3 fatty acids at the right time to assure adequate intake of said multivitamins, minerals, coenzyme, amino acids and omega-3 fatty acids which can be used for prophylactic or supportive use as an adjuvant to anticonvulsant medicines used for treating/preventing of various types of epilepsy or seizures.

The inventors of the present invention also found that a novel pharmaceutical or nutraceutical composition for oral administration comprising multivitamins, minerals, coenzyme, amino acids and omega-3 fatty acids of high quality and purity exhibit qualities such as but not limited to stability, easy administration, easy dose adjustment, minimum side effects, high therapeutic efficiency, and better patient compliance, wherein said composition is indicated for its prophylactic or supportive use as an adjuvant to anticonvulsant medicines used for treating/preventing of various types of epilepsy or seizures.

### OBJECTS OF THE INVENTION

It is an object of the present invention to provide a novel pharmaceutical or nutraceutical composition comprising multivitamins, minerals, coenzyme, amino acid and omega-3 fatty acids and one or more suitable pharmaceutically acceptable excipient.

It is an object of the present invention to provide a novel oral pharmaceutical or nutraceutical composition comprising multivitamins, minerals, coenzyme, amino acid and omega-3 fatty acids and one or more suitable pharmaceutically acceptable excipient.

It is an object of the present invention to provide a novel oral pharmaceutical or nutraceutical composition, comprising:
a) One or more vitamin;
b) One or more mineral;
c) One or more coenzyme;
d) One or more amino acid;
e) One or more omega-3 fatty acid; and
f) One or more pharmaceutically acceptable excipient.

It is an object of the present invention to provide a novel oral pharmaceutical or nutraceutical composition, comprising:
a) 0.001 mg to 50 mg of One or more vitamin;
b) 0.001 mg to 500 mg of One or more mineral;
c) 1 mg to 200 mg of One or more coenzyme;
d) 1 mg to 800 mg of One or more amino acid;
e) 1 mg to 1600 mg of One or more omega-3 fatty acid; and
f) One or more pharmaceutically acceptable excipient.

It is an object of the present invention to provide a novel oral pharmaceutical or nutraceutical composition, comprising:
a) 0.001 mg to 20 mg of One or more vitamin;
b) 0.001 mg to 500 mg of One or more mineral;
c) 10.0 mg to 150 mg of One or more coenzyme;
d) 200 mg to 500 mg of One or more amino acid;
e) 500 mg to 1000 mg of One or more omega-3 fatty acid; and
f) One or more pharmaceutically acceptable excipient.

It is an object of the present invention to provide a novel oral pharmaceutical or nutraceutical composition comprising:
a) One or more vitamin selected from a group consisting of Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K or combinations thereof;
b) One or more mineral selected from a group consisting of calcium, phosphorus, potassium, sodium, magnesium, sulfur, iron, chlorine, cobalt, copper, zinc, manganese, molybdenum, iodine, selenium, chromium or combinations thereof;
c) One or more coenzyme such as but not limited to coenzyme Q10 (ubiquinone);
d) One or more amino acid selected from a group consisting of taurine (tauric acid), leucine, arginine, glutamate, glycine or combinations thereof;
e) One or more Omega-3 fatty acids selected from a group consisting of docosahexaenoic acid (DHA), Alpha-linolenic acid (ALA) and eicosapentaenoic acid (EPA), or combinations thereof; and
f) One or more suitable pharmaceutically acceptable excipient.

It is an object of the present invention to provide a novel oral pharmaceutical or nutraceutical composition, comprising:
a) 0.001 mg to 5.0 mg of Vitamin B6 (Pyridoxine hydrochloride);
b) 1.0 mcg to 400 mcg of Vitamin B9 (Folic acid);
c) 1.0 mcg to 5.0 mcg of Vitamin B12;
d) 1 mcg to 30 mcg of Vitamin D3 (Cholecalciferol);
e) 1 mg to 20 mg of Vitamin E (Tocopherol);
f) 1 mg to 30 mg of Zinc;
g) 1 mg to 500 mg of Magnesium;
h) 1 mg to 10 mg of Manganese;
i) 1 mcg to 80 mcg of Selenium;
j) 1 mg to 1600 mg of Omega 3 fatty acid;
k) 1 mg to 200 mg of Co-enzyme Q10;
l) 1 mg to 800 mg of Taurine (2-aminoethanesulfonic acid); and
m) One or more pharmaceutically acceptable excipient.

It is an object of the present invention to provide a novel oral pharmaceutical or nutraceutical composition, comprising:
a) 0.001 mg to 3.5 mg of Vitamin B6 (Pyridoxine hydrochloride);
b) 25 mcg to 350 mcg of Vitamin B9 (Folic acid);
c) 1.0 mcg to 2.2 mcg of Vitamin B12;
d) 1.0 mcg to 20 mcg of Vitamin D3 (Cholecalciferol);
e) 1.0 mg to 15 mg of Vitamin E (Tocopherol);
f) 1.0 mg to 20 mg of Zinc;
g) 1.0 mg to 500 mg of Magnesium;
h) 1.0 mg to 5.0 mg of Manganese;
i) 1.0 mcg to 50 mcg of Selenium;
j) 1.0 mg to 1000 mg of Omega 3 fatty acid;
k) 1.0 mg to 150 mg of Co-enzyme Q10;
l) 1.0 mg to 500 mg of Taurine (2-aminoethanesulfonic acid); and
m) One or more pharmaceutically acceptable excipient.

It is another object of the present invention to provide a process of preparation of a novel oral pharmaceutical or nutraceutical composition, comprises steps of:
a) Dissolving multivitamins, minerals, coenzyme, amino acid and omega-3 fatty acids into suitable solvent or vehicle to obtain solution;
b) Adding and dissolving one or more suitable pharmaceutically acceptable excipient into solution of step (a);
c) Filtering solution of step (b) through suitable filter; and
d) Filling into suitable bottle or container.

It is another object of the present invention to provide a process of preparation of a novel oral pharmaceutical or nutraceutical composition, comprises steps of:
a) Sifting multivitamins, minerals, coenzyme, amino acid and omega-3 fatty acids through suitable sieve;
b) Sifting one or more suitable pharmaceutically acceptable excipient through suitable sieve;
c) pre-lubricating sifted material of step (a) and step (b) and blending into suitable blender;
d) Lubricating above material and blending it for suitable time; and
e) Compressing blend of step (d) into suitable dosage form.

It is another object of the present invention to provide a process of preparation of a novel oral pharmaceutical or nutraceutical composition, comprises steps of:
a) Sifting multivitamins, minerals, coenzyme, amino acid and omega-3 fatty acids through suitable sieve;
b) Dry mixing of components of step (a);
c) Preparing binder solution;
d) Granulating mixture obtained in step (b) using binder solution obtained in step (c);
e) Drying the granules obtained in step (d);
f) Sifting, blending and lubricating the dried granules of step (e); and
g) Compressing granules of step (f) into suitable dosage form.

It is another object of the present invention to provide a method of using a novel oral pharmaceutical or nutraceutical composition, comprising multivitamins, minerals, coenzyme, amino acid and omega-3 fatty acids for prophylactic or supportive use as an adjuvant to anticonvulsant medicines used for treating/preventing of various types of epilepsy or seizures in all age groups of patients.

### SUMMARY OF THE INVENTION

In one aspect, the present invention discloses a novel pharmaceutical or nutraceutical composition comprising multivitamins, minerals, coenzyme, amino acids and omega-3 fatty acids and one or more suitable pharmaceutically acceptable excipient.

In one aspect, the present invention relates to a novel oral pharmaceutical or nutraceutical composition comprising multivitamins, minerals, coenzyme, amino acids and omega-3 fatty acids and one or more suitable pharmaceutically acceptable excipient.

In another aspect, the present invention provides a novel oral pharmaceutical or nutraceutical composition, comprising:
a) One or more vitamin;
b) One or more mineral;
c) One or more coenzyme;
d) One or more amino acid;
e) One or more omega-3 fatty acid; and
f) One or more pharmaceutically acceptable excipient.

In another aspect, the present invention provides a novel oral pharmaceutical or nutraceutical composition, comprising:
a) 0.001 mg to 50 mg of One or more vitamin;
b) 0.001 mg to 500 mg of One or more mineral;
c) 1 mg to 200 mg of One or more coenzyme;
d) 1 mg to 800 mg of One or more amino acid;
e) 1 mg to 1600 mg of One or more omega-3 fatty acid; and
f) One or more pharmaceutically acceptable excipient.

In another aspect, the present invention provides a novel oral pharmaceutical or nutraceutical composition, comprising:
a) 0.001 mg to 20 mg of One or more vitamin;
b) 0.001 mg to 500 mg of One or more mineral;
c) 10.0 mg to 150 mg of One or more coenzyme;
d) 200 mg to 500 mg of One or more amino acid;
e) 500 mg to 1000 mg of One or more omega-3 fatty acid; and
f) One or more pharmaceutically acceptable excipient.

In another aspect, the present invention discloses a novel oral pharmaceutical or nutraceutical composition comprising:
a) One or more vitamin selected from a group consisting of Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K and combinations thereof;
b) One or more mineral selected from a group consisting of calcium, phosphorus, potassium, sodium, magnesium, sulfur, iron, chlorine, cobalt, copper, zinc, manganese, molybdenum, iodine, selenium, chromium and combinations thereof;
c) One or more coenzyme such as but not limited to coenzyme Q10 (ubiquinone);
d) One or more amino acid selected from a group consisting of taurine (tauric acid), leucine, arginine, glutamate, glycine and combinations thereof;
e) One or more Omega-3 fatty acids selected from a group consisting of docosahexaenoic acid (DHA), Alpha-linolenic acid (ALA) and eicosapentaenoic acid (EPA), and combinations thereof; and
f) One or more pharmaceutically acceptable excipient.

In another aspect, the present invention discloses a novel oral pharmaceutical or nutraceutical composition, comprising:
a) 0.001 mg to 5.0 mg of Vitamin B6 (Pyridoxine hydrochloride);
b) 1.0 mcg to 400 mcg of Vitamin B9 (Folic acid);
c) 1.0 mcg to 5.0 mcg of Vitamin B12;
d) 1 mcg to 30 mcg of Vitamin D3 (Cholecalciferol);
e) 1 mg to 20 mg of Vitamin E (Tocopherol);
f) 1 mg to 30 mg of Zinc;
g) 1 mg to 500 mg of Magnesium;
h) 1 mg to 10 mg of Manganese;
i) 1 mcg to 80 mcg of Selenium;
j) 1 mg to 1600 mg of Omega 3 fatty acid;
k) 1 mg to 200 mg of Co-enzyme Q10;
l) 1 mg to 800 mg of Taurine (2-aminoethanesulfonic acid); and
m) One or more suitable pharmaceutically acceptable excipient.

In another aspect, the present invention discloses a novel oral pharmaceutical or nutraceutical composition, comprising:
a) 0.001 mg to 3.5 mg of Vitamin B6 (Pyridoxine hydrochloride);
b) 25 mcg to 350 mcg of Vitamin B9 (Folic acid);
c) 1.0 mcg to 2.2 mcg of Vitamin B12;
d) 1.0 mcg to 20 mcg of Vitamin D3 (Cholecalciferol);
e) 1.0 mg to 15 mg of Vitamin E (Tocopherol);
f) 1.0 mg to 20 mg of Zinc;
g) 1.0 mg to 500 mg of Magnesium;
h) 1.0 mg to 5.0 mg of Manganese;
i) 1.0 mcg to 50 mcg of Selenium;
j) 1.0 mg to 1000 mg of Omega 3 fatty acid;
k) 1.0 mg to 150 mg of Co-enzyme Q10;
l) 1.0 mg to 500 mg of Taurine (2-aminoethanesulfonic acid); and
m) One or more suitable pharmaceutically acceptable excipient.

In another aspect, the present invention also relates to a process of preparation of a novel pharmaceutical or nutraceutical composition comprising multivitamins, minerals, coenzyme, amino acids and omega-3 fatty acids and one or more suitable pharmaceutically acceptable excipient.

In another aspect, the present invention provides a process of preparation of a novel oral pharmaceutical or nutraceutical composition, comprises steps of:
a) Dissolving multivitamins, minerals, coenzyme, amino acid and omega-3 fatty acids into suitable solvent or vehicle to obtain solution;
b) Adding and dissolving one or more suitable pharmaceutically acceptable excipient into solution of step (a);
c) Filtering solution of step (b) through suitable filter; and
d) Filling into suitable bottle or container.

In another aspect, the present invention provides a process of preparation of a novel oral pharmaceutical or nutraceutical composition, comprises steps of:
a) Sifting multivitamins, minerals, coenzyme, amino acid and omega-3 fatty acids through suitable sieve;
b) Sifting one or more suitable pharmaceutically acceptable excipient through suitable sieve;
c) pre-lubricating sifted material of step (a) and step (b) and blending into suitable blender;
d) Lubricating above material and blending it for suitable time; and
e) Compressing blend of step (d) into suitable dosage form.

In another aspect, the present invention provides a process of preparation of a novel oral pharmaceutical or nutraceutical composition comprises steps of:
a) Sifting multivitamins, minerals, coenzyme, amino acid and omega-3 fatty acids through suitable sieve;
b) Dry mixing of components of step (a);
c) Preparing binder solution;
d) Granulating mixture obtained in step (b) using binder solution obtained in step (c);
e) Drying the granules obtained in step (d);
f) Sifting, blending and lubricating the dried granules of step (e); and
g) Compressing granules of step (f) into suitable dosage form.

In another aspect, the present invention discloses a method of using a novel oral pharmaceutical or nutraceutical composition comprising multivitamins, minerals, coenzyme, amino acid and omega-3 fatty acids for prophylactic or supportive use as an adjuvant to anticonvulsant medicines used for treating/preventing of various types of epilepsy or seizures in all age groups of patients.

### DETAILED DESCRIPTION OF THE INVENTION

The term “composition” or “pharmaceutical composition” or “pharmaceutical composition for oral administration” or “stable composition for oral administration” or “nutraceutical composition” as used herein synonymously include dosage forms such as tablet, capsule, effervescent tablet, chewable tablet, pill, granules, powder, pellets, oral syrup, oral solution, oral suspension or the like.

Preferably, the pharmaceutical composition refers to tablet, capsule, effervescent tablet, chewable tablet, oral syrup and oral solution.

The term “composition”, as in pharmaceutical or nutraceutical composition, is intended to encompass a drug product/nutrient comprising multivitamins, minerals, coenzyme, amino acids and omega-3 fatty acids and other inert ingredient(s) (pharmaceutically acceptable excipients).

The term “nutraceutical composition” comprises comprising multivitamins, minerals, coenzyme, amino acids and omega-3 fatty acids and other inert ingredient(s) (pharmaceutically acceptable excipients).

The present invention relates to a novel pharmaceutical or nutraceutical composition comprising multivitamins, minerals, coenzyme, amino acids, omega-3 fatty acids and one or more pharmaceutically acceptable excipient.

The term “multivitamins” is used in broad sense to include combination different types of vitamins.

The term “minerals” is used in broad sense to include one or more types of minerals.

The term “amino acids” is used in broad sense to include one or more types of amino acids such as but not limited to taurine (tauric acid), leucine, arginine, glutamate, glycine or the like.

The term “omega-3 fatty acids” is used in broad sense to include one or more types of omega-3 fatty acids such as but not limited to α-linolenic acid (ALA), eicosapentaenoic acid (EPA) and docosahexaenoic acid (DHA) or the like.

The term “stable” as used herein refers to formulations that substantially retain the label amount of the therapeutically active ingredient during storage for commercially relevant times, and the drug-related impurity contents in the formulations remain within the acceptable limit.

The term “treat”, “treating” or “treatment” refers to obtaining desired pharmacological and/or physiological effect. The effect can be prophylactic or therapeutic, which includes achieving, partially or substantially, one or more of the following results: partially or totally reducing the extent of the disease, disorder or syndrome; ameliorating or improving a clinical symptom or indicator associated with the disorder or delaying, inhibiting or decreasing the likelihood of the progression of the disease, disorder or syndrome, and/or symptoms thereof.

The term “preventing” or “prevention” refers to a procedure through which individuals, particularly those with risk factors for a disease, are treated in order to prevent a disease from occurring.

The term “Epilepsy” is a group of neurological disorders characterized by recurrent epileptic seizures.

The term “seizure” formally known as an epileptic seizure, is a period of symptoms due to abnormally excessive or synchronous neuronal activity in the brain.

The term “Pharmaceutically acceptable excipient(s)” or suitable pharmaceutically acceptable excipient are components that are added to the pharmaceutical or nutraceutical composition other than the active ingredient.

The term “adjuvant” refers to the administration of the composition of present invention, it can be administered simultaneously, sequentially, before or after the administration of anticonvulsant medicines.

The use of the terms “a” and “an” and “the” and similar referents in the context of describing the invention (especially in the context of the following claims) are to be construed to cover both the singular and the plural, unless otherwise indicated herein or clearly contradicted by context.

Throughout this specification and the appended claims, it is to be understood that the words “comprise”, “have”, “contain” and “include” and variations such as “comprises”, “comprising”, “having”, “containing” “includes”, “including” are to be interpreted inclusively, unless the context requires otherwise. That is, the use of these words may imply the inclusion of an element or elements not specifically recited.

Excipients may be added to facilitate manufacture, enhance stability, enhance product characteristics, enhance patient acceptability etc. Pharmaceutically acceptable excipient(s) includes, but not limited to, diluents, binders, disintegrants, lubricants, glidants, antioxidants, fillers, taste masking agents, chelating agent, thickening agent, flavoring agents, coating agents, coloring agents, solvent(s), vehicle, co-solvent(s), solubility enhancers, antiadherents, preservatives, sorbents, sweeteners, release-controlling polymers stabilizing agent, acidic agent, basic agent, effervescent agents, buffering agent, viscosity regulating agents, pH regulating agent and any other excipient known to the art for making pharmaceutical composition.

In one embodiment, the present invention relates to a stable composition for oral administration comprising multivitamins, minerals, coenzyme, amino acids and omega-3 fatty acids according to present invention can be prepared into single dose pharmaceutical composition having immediate release or modified release or controlled release or delayed release or any combination thereof.

The novel pharmaceutical or nutraceutical composition comprising multivitamins, minerals, coenzyme, amino acids and omega-3 fatty acids according to the present invention may be in the form of tablet, effervescent tablet, chewable tablet, modified release tablet, controlled release tablet, delayed release tablet, capsule, granules, pellets, beads or sachet, oral syrup, liquid oral solution, solution or suspension or emulsion or the like.

In another embodiment, the present invention also relates to a process for the preparation of a novel pharmaceutical or nutraceutical composition for oral administration comprising multivitamins, minerals, coenzyme, amino acids and omega-3 fatty acids.

In another embodiment, the present invention comprises one or more vitamin is selected from group consisting of Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K or the like or any combination thereof.

In one embodiment, Vitamin D is a fat-soluble secosteroid that enhances intestinal absorption of calcium. Vitamin D has other roles in the body, including modulation of cell growth, neuromuscular and immune function, and reduction of inflammation. Vitamin D comprises various forms, including the two primary forms in humans, cholecalciferol (vitamin D3) and ergocalciferol (vitamin D2). Vitamin D3 is produced photochemically in the skin upon exposure to sunlight. Vitamin D is also ingested from diet or supplements. Vitamin D is used as a one of the vitamins according to the present invention, and is present in an amount from about 1.0 mcg to about 30 mcg, more preferably from about 1.0 mcg to about 20 mcg with respect to total weight of the pharmaceutical or nutraceutical composition.

In one embodiment, Vitamin E, a fat-soluble vitamin, is an antioxidant vitamin involved in the metabolism of all cells. It protects vitamin A and essential fatty acids from oxidation in the body cells and prevents breakdown of body tissues. Vitamin E is the generic term for a group of related substances that include alpha-tocopherol, beta-tocopherol, gammatocopherol, and delta-tocopherol. In addition, each of these four compounds has a “d” form, which is the natural form, and a “dl” form that is the synthetic form. Preferably, vitamin E is in the natural form. it is present in an amount from about 1.0 mg to about 20 mg of vitamin E; and more preferably from about 1.0 mg to about 15 mg with respect to total weight of the pharmaceutical or nutraceutical composition.

In one embodiment, Vitamin B6 is one of the B vitamins, and thus an essential nutrient. It's active form, pyridoxal 5′-phosphate, serves as a coenzyme in more than 140 enzyme reactions in amino acid, glucose, and lipid metabolism.

Vitamin B6 is a water-soluble vitamin, one of the B vitamins. The vitamin actually comprises a group of six chemically related compounds, i.e., vitamers, that all contain a pyridine ring as their core. These are pyridoxine, pyridoxal, pyridoxamine, and their respective phosphorylated derivatives pyridoxine 5′-phosphate, pyridoxal 5′-phosphate and pyridoxamine 5′-phosphate. Pyridoxal 5′-phosphate has the highest biological activity, but the others are convertible to that form. Vitamin B6 serves as a co-factor in more than 140 cellular reactions, mostly related to amino acid biosynthesis and catabolism, but is also involved in fatty acid biosynthesis and other physiological functions.

Preferably Vitamin B6 (pyridoxine hydrochloride) is present in an amount from about 0.001 mg to about 5.0 mg, more preferably from about 0.001 mg to about 3.5 mg with respect to total weight of the pharmaceutical or nutraceutical composition.

In one embodiment, vitamin B9 refers to the many forms of folic acid and its related compounds, including tetrahydrofolic acid (the active form), methyltetrahydrofolate (the primary form found in blood), methenyltetrahydrofolate, folinic acid, folacin, and pteroylglutamic acid.

Preferably. Vitamin B9 (Folic acid) is present in an amount from about 1.0 mcg to about 400 mcg, more preferably from about 25 mcg to about 350 meg with respect to total weight of the pharmaceutical or nutraceutical composition.

In one embodiment, the Vitamin B12 or Cyanocobalamin is also used to treat pernicious anemia, vitamin B12 deficiency (due to low intake from food or inability to absorb due to genetic or other factors), thyrotoxicosis, hemorrhage, malignancy, liver disease and kidney disease.

Vitamin B12 deficiency has significative impact on the nervous system causing irreversible lesions, with progressive increase of the myelinated neurons, demyelination and death of neuronal cells in the spine and cerebral cortex, with symptoms such as paresthesia of the hands and feet, decreased sensation of vibration and position, with consequent instability, confusion, moodiness and loss of memory and central vision, possibly still exhibiting delirium, hallucinations or even frank psychosis.

Preferably, Vitamin B12 (Cyanocobalamin) is present in an amount from about 1.0 mcg to 5.0 mcg, more preferably from about 1.0 mcg to about 2.2 meg with respect to total weight of the pharmaceutical or nutraceutical composition.

In another embodiment, the present invention comprises one or more minerals selected from group consisting of calcium, phosphorus, potassium, sodium, magnesium, sulfur, iron, chlorine, cobalt, copper, zinc, manganese, molybdenum, iodine, selenium, sodium selenite, chromium or the like or any combination thereof.

In one embodiment, Magnesium is the fourth mineral in greater abundance in ‘body of the human organism and is involved in about 325 enzymatic reactions, including cellular energy production via ATP-Mg complex formation, cell membrane stabilization and nucleic acid, proteins and cytoplasmic organelles synthesis. Therefore, this mineral plays an important role in the electrical stability and integrity of the cell membrane, muscle contraction, nerve conduction, regulation of vascular tone, among others. It has also been associated with anxiety-related disorders. Deficiency (hypomagnesemia) causes nervous system irritability with tetany, fatigue, vasodilation, muscle spasms or cramps, convulsions, tremors, depression and psychotic behavior.

Preferably Magnesium is present in an amount of from about 1.0 mg to about 500 mg with respect to total weight of the pharmaceutical or nutraceutical composition.

In one embodiment, Manganese (Mn), a necessary human micronutrient, is a trace element essential for many physiological processes due to its role in the activity of various metalloprotein enzymes. There are eleven known oxidative states of manganese, of which Mn2+, Mn3+ and Mn4+ are found in humans and animals.

It is found mostly in bones, the liver, kidneys, and pancreas. Manganese helps the body form connective tissue, bones, blood clotting factors, and sex hormones. It also plays a role in fat and carbohydrate metabolism, calcium absorption, and blood sugar regulation. Manganese is also necessary for normal brain and nerve function. Low levels of manganese in the body can contribute to infertility, bone malformation, weakness, and seizures.

Preferably Manganese is present in an amount of from about 1.0 mg to 10 mg, more preferably from about 1.0 mg to 5.0 mg with respect to total weight of the pharmaceutical or nutraceutical composition.

In one embodiment, Zinc is considered an essential nutrient, meaning that your body can't produce or store it. Zinc is an essential mineral that your body does not make on its own. It aids growth, DNA synthesis, immune function and more. Today, approximately 70 enzymes, many of them essential to human wellbeing, have been found to contain zinc, and the evidence is strong that zinc is required for many (if not all) of these enzymes to express their activity.

Preferably Zinc is present in an amount of from about 1.0 mg to about 30 mg, more preferably from about 1.0 mg to about 20 mg with respect to total weight of the pharmaceutical or nutraceutical composition.

In one embodiment, Selenium is a trace mineral, which means that the body only needs a small amount of it. It is found naturally in foods or as a supplement. Selenium is an essential component of various enzymes and proteins, called selenoproteins, that help to make DNA and protect against cell damage and infections; these proteins are also involved in reproduction and the metabolism of thyroid hormones. Most selenium in the body is stored in muscle tissue, although the thyroid gland holds the highest concentration of selenium due to various selenoproteins that assist with thyroid function. Selenium is a component of selenoproteins and enzymes. These have antioxidant properties that help to break down peroxides, which can damage tissues and DNA, leading to inflammation and other health problems.

Preferably, sodium selenite is present in an amount of from about 1.0 mcg to 80 meg, more preferably from about 1.0 mcg to 50 mcg with respect to total weight of the pharmaceutical or nutraceutical composition.

In one embodiment, the present invention comprises one or more amino acid selected from group consisting of taurine (tauric acid), leucine, arginine, glutamate, glycine or the like.

In another embodiment, Taurine (2-aminocthanesulfonic acid) is a conditionally essential amino acid. It has been found free or in simple peptides but is not used in protein synthesis. In vivo studies have shown that taurine is essential in a number of aspects of mammalian development. In addition, low levels of taurine are associated with different pathological lesions, including cardiomyopathy, retinal degeneration, and growth retardation. It is known that taurine plays an important role in numerous physiological functions, including detoxification, membrane stabilization, osmoregulation, and modulation of cellular calcium levels. Taurine has also been used in the treatment of cardiovascular diseases, epilepsy and other seizure disorders, macular degeneration, Alzheimer's disease, liver disorders, and cystic fibrosis.

Preferably, taurine is present in an amount of from about 1.0 mg to about 800 mg, more preferably from about 1.0 mg to 500 mg with respect to total weight of the pharmaceutical or nutraceutical composition.

In one embodiment, present invention comprises one or more Omega-3 fatty acids selected from group consisting of docosahexaenoic acid (DHA), Alpha-linolenic acid (ALA) and eicosapentaenoic acid (EPA) or the like or any combination thereof.

In another embodiment, Omega-3 fatty acids, also called Omega-3 oils, ω-3 fatty acids or n-3 fatty acids, are polyunsaturated fatty acids (PUFAs) characterized by the presence of a double bond, three atoms away from the terminal methyl group in their chemical structure. The three types of omega-3 fatty acids involved in human physiology are α-linolenic acid (ALA), eicosapentaenoic acid (EPA) and docosahexaenoic acid (DHA). ALA can be found in plants, while DHA and EPA are found in algae and fish. Marine algae and phytoplankton are primary sources of omega-3 fatty acids. DHA and EPA accumulate in fish that cat these algae.

Preferably omega-3 fatty acid is present in an amount from about 1.0 mg to about 1600 mg, more preferably from about 1.0 mg to 1000 mg with respect to total weight of the pharmaceutical or nutraceutical composition.

In one embodiment, present invention comprises one or more coenzyme such as but not limited to coenzyme Q10 (ubiquinone) or the like.

In another embodiment, Coenzyme Q, also known as ubiquinone, is a coenzyme family that is ubiquitous in animals and most bacteria (hence the name ubiquinone). In humans, the most common form is coenzyme Q10 or ubiquinone-10. It is a 1,4-benzoquinone, where Q refers to the quinone chemical group and 10 refers to the number of isoprenyl chemical subunits in its tail. In natural ubiquinones, the number can be anywhere from 6 to 10. This family of fat-soluble substances. There are three redox states of CoQ: fully oxidized (ubiquinone), semiquinone (ubisemiquinone), and fully reduced (ubiquinol). The capacity of this molecule to act as a two-electron carrier (moving between the quinone and quinol form) and a one-electron carrier (moving between the semiquinone and one of these other forms) is central to its role in the electron transport chain due to the iron-sulfur clusters that can only accept one electron at a time, and as a free-radical-scavenging antioxidant.

Preferably, coenzyme Q10 is present in an amount from about 1.0 mg to about 200 mg, more preferably from about 1.0 mg to 150 mg with respect to total weight of the pharmaceutical composition.

The pharmaceutically acceptable excipients are selected from the group consisting of diluents, binders, disintegrants, lubricants, glidants, filler, preservative, antioxidants, taste masking agents, chelating agent, thickening agent, flavoring agents, coating agents, coloring agents, solvent(s), cosolvent(s), vehicle, antiadherents, preservatives, sorbents, sweeteners, release-controlling polymers stabilizing agent, acidic agent, basic agent, buffering agent, effervescent agent, pH regulating agent and any other excipient known to the art for making pharmaceutical composition.

The examples of vehicles according to the present invention include but not limited to group comprising of sorbitol, water, purified water, polyhydric alcohols, hydro-alcoholic solutions and buffers or oily (e.g., vegetable or mineral oils, organic oily bases, emulsified bases etc), Polyoxyl 40 hydrogenated castor oil (kolliphor RH 40), monopalmitate (Tween 60), sodium oleate, polyoxyethylene stearate, potassium oleate and combination thereof.

The examples of solvents according to the present invention include but not limited to group comprising of aqueous or inert organic solvents or inorganic acids, alcohols, ketones, esters, ethers, aliphatic hydrocarbons, halogenated solvents, cycloaliphatic solvents, aromatic solvents, heterocyclic solvents, and mixtures thereof. Typical solvents include water, purified water, glycerin, acetone, diacetone alcohol, methanol, ethanol, isopropyl alcohol, butyl alcohol, ethylene glycol monoethyl ether, monoethyl acetate, cyclohexane, benzene, toluene, naphtha, 1,4-dioxane, tetrahydrofuran, hydrochloric acid (HCl), aqueous solvents containing inorganic salts (such as sodium chloride, calcium chloride, and the like), and mixtures thereof (such as acetone and water, acetone and methanol, acetone and ethyl alcohol, methylene dichloride and methanol, and ethylene dichloride and methanol) or mixture thereof.

The examples of the co-solvent according to the present invention include but not limited to group comprising of propylene glycol, chloroform, ethanol glycerine, polyethylene glycol and combination thereof.

The examples of the preservatives according to the present invention include but not limited to group comprising of bronopol, methyl hydroxybenzoate, propyl hydroxybenzoate, sodium benzoate, benzoic acid, phenethyl alcohol, benzyl alcohol, methyl paraben, methyl paraben sodium, propyl paraben sodium, imidazolidinylurea, p-hydroxybenzoate esters, chlorobutanol, dehydroacetic acid, benzalkonium chloride, merfen, thiomersal, cetyltrimethylammonium bromide, cetylpyridinium chloride, sorbic acid or mixtures thereof.

The examples of the buffer according to the present invention include but not limited to a group comprising of tartaric acid, citric acid, citric acid monohydrate, malic acid, acetic acid, glacial acetic acid, lactic acid, sodium citrate and disodium hydrogen phosphate.

The examples of sweeteners according to the present invention include but not limited to a group comprising of aspartame, acesulfame potassium, aspartame acesulfame, glycerin, dipotassium glycyrrhizinate, stevia, thaumatin, saccharose, glucose, maltose, galactose, and artificial sweeteners, such as, sodium saccharin, cyclamates, sucralose, sodium saccharinate, neohesperidine dihydrochalcone, monoammonium glycyrrhizinate, dextrose, invert sugar, fructose, Stevia rebaudiana (Stevioside); sugar alcohols such as sorbitol, mannitol, xylitol, and the like. Also contemplated are hydrogenated starch hydrolysates and the synthetic sweetener and sodium and calcium salts thereof, and natural intensive sweeteners, or mixture thereof.

The examples of the chelating agent according to the present invention include but not limited to a group comprising of sodium EDTA, disodium EDTA.

The examples of the thickening agent according to the present invention include but not limited to a group comprising of xanthan gum, PVPK-30, PVPK-90, PEG 400, hydroxyethyl cellulose, methyl cellulose, sodium Carboxymethyl cellulose, Hydroxy propyl cellulose, Hydroxyl propyl methylcellulose K4 M, K100 M, and a water-soluble Carboxyvinyl polymer.

The examples of taste masking agent according to the present invention include but not limited to a group comprising of Hydroxypropyl betadex, vanilla, grape, lemon, lime, orange, cherry, grapefruit, berries, mints, licorice, coffee, chocolate, mint, peach, raspberry or combination thereof.

The examples of the antioxidant according to the present invention include but not limited to a group comprising of butylated hydroxytoluene, butylated hydroxyanisole, DL-alpha-tocopherol, propyl gallate, octylgallate, ascorbyl palmitate, acetyl cysteine, ascorbic acid, sodium ascorbate, fumaric acid, lecithin, sodium metabisulfite, sodium sulfite or combination thereof.

The examples of flavoring agent according to the present invention include but not limited to a group comprising of fruit flavor, peppermint flavor, lemon, lemon-lime, orange, sour cherry, mint, honey vanilla, citrus oil, grapefruit grape, menthol, cranberry, vanilla berry, bubble gum, cherry, fruit essences including apple, pear, peach, strawberry, raspberry, plum, pineapple, apricot or other fruit flavors or combination thereof.

The examples of the colourant according to the present invention include but not limited to a group comprising of quinolone yellow, sunset yellow, erythrosine, brilliant blue or the like.

The examples of diluent(s) or filler according to the present invention include but not limited to a group comprising of Emdex/dextrates, citric acid, mannitol, sorbitol, sucrose, microcrystalline cellulose, carboxymethyl cellulose (carmellose), sodium carboxymethyl cellulose (carmellose sodium), corn starch, potato stach, anhydrous lactose, lactose monohydrate or combination thereof.

The examples of acidic agents or acidifying agents according to the present invention include but not limited to a group comprising of citric acid maleic acid, tartaric acid, fumaric acid, adipic acid, succinic acid, lactic acid, glycolic acid, alpha-hydroxy acids, ascorbic acid and amino acids, anhydrides, salts and derivatives of these acids or combination thereof.

The examples of basic agents according to the present invention include but not limited to a group comprising of potassium carbonate, sodium carbonate, sodium hydrogen carbonate, sodium bicarbonate, potassium hydrogen carbonate, arginine carbonate, glycine carbonate or lithium carbonate, calcium carbonate, ammonium carbonate, L-lysine carbonate, sodium glycine carbonate, sodium carbonates of amino acids, anhydrous sodium perborate, effervescent perborate, sodium perborate monohydrate, sodium percarbonate, sodium dichloroisocyanurate, sodium hypochlorite, calcium hypochlorite, tris, tartrate, acetate, phosphate or mixture thereof.

The examples of lubricant according to the present invention include but not limited to a group comprising of calcium stearate, magnesium stearate, sodium stearyl fumarate, fumaric acid, calcium stearate, polyethylene glycol, sodium benzoate, potassium benzoate, sodium lauryl sulphate, talc, colloidal silica, sucrose fatty acid esters, stearic acid, zinc stearate, hydrogenated vegetable oil, mineral oil, glyceryl behenate, polyoxyethylene glycols, sodium benzoate, dimethicone or liquid paraffin, or mixture thereof.

Binders according to the present invention include, but not limited to a group comprising of pregelatinized starch; partially pregelatinized starch; cellulose or a derivative thereof such as microcrystalline cellulose, methyl cellulose, ethyl cellulose, sodium carboxymethyl cellulose (carmellose sodium), hydroxyethyl cellulose, hydroxyethyl methyl cellulose, hydroxypropyl methylcellulose xanthan gum, cellulose gum, guar gum, pullulan, gum arabic, acacia powder, polyvinyl pyrrolidone, polyacrylamides, polyvinyloxoazolidone, polyvinyl alcohols, or combination thereof.

The examples of glidant according to the present invention include but not limited to a group comprising of colloidal silica, colloidal silicone dioxide, pyrogenic silica, hydrated sodium silicoaluminate, magnesium stearate, sodium stearyl fumarate, starch, talc, calcium phosphate tribasic, magnesium oxide; magnesium silicate, magnesium trisilicate, stearic acid, calcium stearate or mixture thereof.

In one embodiment, the present invention provides a novel pharmaceutical or nutraceutical composition, comprising:
a) One or more vitamin;
b) One or more mineral;
c) One or more coenzyme;
d) One or more amino acid;
e) One or more omega-3 fatty acid; and
f) One or more suitable pharmaceutically acceptable excipient.

In one embodiment, the present invention provides a novel pharmaceutical or nutraceutical composition for oral administration, comprising:
a) One or more vitamin;
b) One or more mineral;
c) One or more coenzyme;
d) One or more amino acid;
e) One or more omega-3 fatty acid; and
f) One or more suitable pharmaceutically acceptable excipient.

In one embodiment, the present invention provides a novel oral pharmaceutical or nutraceutical composition, comprising:
a) 0.001 mg to 50 mg of One or more vitamin;
b) 0.001 mg to 500 mg of One or more mineral;
c) 1 mg to 200 mg of One or more coenzyme;
d) 1 mg to 800 mg of One or more amino acid;
e) 1 mg to 1600 mg of One or more omega-3 fatty acid; and
f) One or more suitable pharmaceutically acceptable excipient.

In one embodiment, the present invention provides a novel oral pharmaceutical or nutraceutical composition, comprising:
a) 0.001 mg to 20 mg of One or more vitamin;
b) 0.001 mg to 500 mg of One or more mineral;
c) 10.0 mg to 150 mg of One or more coenzyme;
d) 200 mg to 500 mg of One or more amino acid;
e) 500 mg to 1000 mg of One or more omega-3 fatty acid; and
f) One or more suitable pharmaceutically acceptable excipient.

In one embodiment, the present invention provides a novel oral pharmaceutical or nutraceutical composition, comprising:
a) One or more vitamin selected from group comprising of Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K and combinations thereof;
b) One or more mineral selected from group comprising of calcium, phosphorus, potassium, sodium, magnesium, sulfur, iron, chlorine, cobalt, copper, zinc, manganese, molybdenum, iodine, selenium, chromium and combinations thereof;
c) One or more coenzyme such as but not limited to coenzyme Q10 (ubiquinone);
d) One or more amino acid selected from group comprising of taurine (tauric acid), leucine, arginine, glutamate, glycine and combinations thereof;
e) One or more Omega-3 fatty acids selected from group comprising of docosahexaenoic acid (DHA), Alpha-linolenic acid (ALA) and eicosapentaenoic acid (EPA), and combinations thereof; and
f) One or more suitable pharmaceutically acceptable excipient.

In one embodiment, the present invention provides a novel oral pharmaceutical or nutraceutical composition, comprising:
a) 0.001 mg to 5.0 mg of Vitamin B6 (Pyridoxine hydrochloride);
b) 1.0 mcg to 400 mcg of Vitamin B9 (Folic acid);
c) 1.0 mcg to 5.0 mcg of Vitamin B12;
d) 1 mcg to 30 mcg of Vitamin D3 (Cholecalciferol);
e) 1 mg to 20 mg of Vitamin E (Tocopherol);
f) 1 mg to 30 mg of Zinc;
g) 1 mg to 500 mg of Magnesium;
h) 1 mg to 10 mg of Manganese;
i) 1 mcg to 80 mcg of Selenium;
j) 1 mg to 1600 mg of Omega 3 fatty acid;
k) 1 mg to 200 mg of Co-enzyme Q10;
l) 1 mg to 800 mg of Taurine (2-aminoethanesulfonic acid); and
m) One or more suitable pharmaceutically acceptable excipient.

In one embodiment, the present invention provides a novel oral pharmaceutical or nutraceutical composition, comprising:
a) 0.001 mg to 3.5 mg of Vitamin B6 (Pyridoxine hydrochloride);
b) 25 mcg to 350 mcg of Vitamin B9 (Folic acid);
c) 1.0 mcg to 2.2 mcg of Vitamin B12;
d) 1.0 mcg to 20 mcg of Vitamin D3 (Cholecalciferol);
e) 1.0 mg to 15 mg of Vitamin E (Tocopherol);
f) 1.0 mg to 20 mg of Zinc;
g) 1.0 mg to 500 mg of Magnesium;
h) 1.0 mg to 5.0 mg of Manganese;
i) 1.0 mcg to 50 mcg of Selenium;
j) 1.0 mg to 1000 mg of Omega 3 fatty acid;
k) 1.0 mg to 150 mg of Co-enzyme Q10;
l) 1.0 mg to 500 mg of Taurine (2-aminoethanesulfonic acid); and
m) One or more suitable pharmaceutically acceptable excipient.

In one embodiment, the present invention also relates to a process of preparation of a novel pharmaceutical or nutraceutical composition comprising multivitamins, minerals, coenzyme, amino acids and omega-3 fatty acids and one or more suitable pharmaceutically acceptable excipient.

In one embodiment, the present invention provides a process of preparation of a novel oral pharmaceutical or nutraceutical composition comprises steps of:
a) Dissolving multivitamins, minerals, coenzyme, amino acid and omega-3 fatty acids into suitable solvent or vehicle to obtain solution;
b) Adding and dissolving one or more suitable pharmaceutically acceptable excipient into solution of step (a);
c) Filtering solution of step (b) through suitable filter; and
d) Filling into suitable bottle or container.

In one embodiment, the present invention provides a process of preparation of a novel oral pharmaceutical or nutraceutical composition, comprises steps of:
a) Sifting multivitamins, minerals, coenzyme, amino acid and omega-3 fatty acids through suitable sieve;
b) Sifting one or more suitable pharmaceutically acceptable excipient through suitable sieve;
c) pre-lubricating sifted material of step (a) and step (b) and blending into suitable blender;
d) Lubricating above material and blending it for suitable time; and
e) Compressing blend of step (d) into suitable dosage form.

In one embodiment, the present invention provides a process of preparation of a novel oral pharmaceutical or nutraceutical composition comprises steps of:
a) Sifting multivitamins, minerals, coenzyme, amino acid and omega-3 fatty acids through suitable sieve;
b) Dry mixing of components of step (a);
c) Preparing binder solution;
d) Granulating mixture obtained in step (b) using binder solution obtained in step (c);
e) Drying the granules obtained in step (d);
f) Sifting, blending and lubricating the dried granules of step (e); and
g) Compressing granules of step (f) into suitable dosage form.

In another embodiment, the present invention discloses a method of using a novel pharmaceutical or nutraceutical composition comprising multivitamins, minerals, coenzyme, amino acid and omega-3 fatty acids for prophylactic or supportive use as an adjuvant to anticonvulsant medicines used for treating/preventing of various types of epilepsy or seizures in all age groups of patients.

In another embodiment, the present invention discloses a method of using a novel oral pharmaceutical or nutraceutical composition comprising multivitamins, minerals, coenzyme, amino acid and omega-3 fatty acids for prophylactic or supportive use as an adjuvant to anticonvulsant medicines used for treating/preventing of various types of epilepsy or seizures in all age groups of patients.

In another embodiment, the present invention discloses a novel oral pharmaceutical or nutraceutical composition comprising multivitamins, minerals, coenzyme, amino acid and omega-3 fatty acids can be used for prophylactic or supportive use when used as an adjuvant to anticonvulsant medicines such as but not limited to Brivaracetam, Carbamazepine, Clobazam, Clonazepam, Diazepam, Ethosuximide, Gabapentin, Lacosamide, Lamotrigine, Levetiracetam, Midazolam, Oxcarbazepine, Perampanel, Phenobarbitone, Phenytoin, Pregabalin, Primidone, Rufinamide, Sodium valproate, Tiagabine, Topiramate, Vigabatrin, Zonisamide or any combination thereof.

The following examples serve to illustrate the embodiments of the present invention.

However, they do not intend to limit the scope of the present invention.

### Example 1: Omega-3 Fatty, Co-Enzyme Q10, Taurine with Multivitamin & Multimineral Syrup

| | | Quantity in mg/mL | |
|---|---|---|---|
| No | Ingredients | (Per 20 mL) | |
| 1 | Omega 3 | 800 | mg |
| 2 | Co-enzyme (CoQ10) | 100 | mg |
| 3 | Taurine (2-aminoethanesulfonic acid) | 400 | mg |
| 4 | Vitamin B6 (Pyridoxine hydrochloride) | 2.4 | mg |
| 5 | Vitamin B9 (Folic acid) | 300 | mcg |
| 6 | Vitamin B12 (Cyanocobalamin) | 2.2 | mcg |
| 7 | Vitamin D3 (cholecalciferol) | 15 | mcg |
| 8 | Vitamin E (tocopherol) | 10 | mg |
| 9 | Zinc | 17 | mg |
| 10 | Magnesium | 300 | mg |
| 11 | Manganese | 2 | mg |
| 12 | Sodium Selenite | 40 | mcg |
| | Inactive Ingredients | | |
| 13 | Sodium Benzoate | 0.04 | mg |
| 14 | Citric acid | 0.01 | mg |
| 15 | Aspartame | 0.01 | mg |
| 16 | Sodium EDTA | 0.01 | mg |
| 17 | Methyl paraben | 0.34 | mg |
| 18 | Propyl paraben | 0.06 | mg |

q.s.: Quantity sufficient

### Manufacturing Process:

1. Dissolve the Active serial number 1-12 into vehicle or solvent and stirred for 15 minutes with 250 RPM
2. Add and dissolve the all remaining inactive ingredients serial number 13-18 into above solution
3. Filter the solution through PES 5 μm filter
4. Filled into 200 mL amber coloured PET bottle.

### Example 2: Omega-3 Fatty, Co-Enzyme Q10, Taurine with Multivitamin & Multimineral Solution

| | | Quantity in mg/mL | |
|---|---|---|---|
| No | Ingredients | (per 20 mL) | |
| 1. | Omega 3 | 800 | mg |
| 2. | Co-enzyme (CoQ10) | 100 | mg |
| 3. | Taurine (2-aminoethanesulfonic acid) | 400 | mg |
| 4. | Vitamin B6 (Pyridoxine hydrochloride) | 2.4 | mg |
| 5. | Vitamin B9 (Folic acid) | 300 | mcg |
| 6. | Vitamin B12 (Cyanocobalamin) | 2.2 | mcg |
| 7. | Vitamin D3 (cholecalciferol) | 15 | mcg |
| 8. | Vitamin E (tocopherol) | 10 | mg |
| 9. | Zinc | 17 | mg |
| 10. | Magnesium | 300 | mg |
| 11. | Manganese | 2 | mg |
| 12. | Sodium Selenite | 40 | mcg |
| | Inactive Ingredients | | |
| 13. | Acesulfame potassium | q.s. | |
| 14. | Aspartame acesulfame | q.s. | |
| 15. | Hydroxypropyl betadex | 3000 | mg |

| | | |
|---|---|---|
| 16. | Sodium benzoate | 0.02 |
| 17. | Sodium metabisulfite | 0.02 |
| 18. | Propylene glycol | q.s. |
| 19. | Purified water | q.s. |

q.s.: Quantity sufficient

### Manufacturing Process:

1. Dissolve the Active of serial number 1-12 into Propylene glycol with water and Hydroxypropyl betadex. and stirred for 30 minutes with 500 RPM;
2. Add and dissolve the all remaining inactive ingredients into above solution;
3. Filter the solution through PES 5 μm filter; and
4. Filled into 250 mL amber coloured PET bottle.

### Example 3: Omega-3 Fatty, Co-Enzyme Q10, Taurine with Multivitamin & Multi-Mineral Effervescent Tablet (by Direct Compression)

| | | Concentration | |
|---|---|---|---|
| No. | Ingredients (Specific form) | (per tablet) | |
| 1. | Omega 3 | 800 | mg |
| 2. | Co-enzyme (CoQ10) | 100 | mg |
| 3. | Taurine (2-aminoethanesulfonic acid) | 400 | mg |
| 4. | Vitamin B6 (Pyridoxine | 2.4 | mg |
| | hydrochloride) | | |
| 5. | Vitamin B9 (Folic acid) | 300 | mcg |
| 6. | Vitamin B12 (Cyanocobalamin) | 2.2 | mcg |
| 7. | Vitamin D3 (cholecalciferol) | 15 | mcg |
| 8. | Vitamin E (tocopherol) | 10 | mg |
| 9. | Zinc | 17 | mg |
| 10. | Magnesium | 300 | mg |
| 11. | Manganese | 2 | mg |
| 12. | Sodium Selenite | 40 | mcg |

Inactive Ingredients

| | | | |
|---|---|---|---|
| 13. | Emdex (Dextrates) | 479.28 | mg |
| 14. | Citric acid Monohydrate | 150 | mg |
| 15. | Sodium Hydrogen carbonate | 150 | mg |
| 16. | Aspartame | 35 | mg |
| 17. | Orange Flavor | 15 | mg |
| 18. | PRUV (Sodium Stearyl Fumarate) | 5 | mg |

| Total Tablet Weight | Around |
|---|---|
| | 2800 mg |

### Manufacturing Process:

1. Sifting the all Active of serial number 1-12 with diluents (dextrates) by cosifting with #30 sieve;
2. Citric acid, sodium hydrogen carbonate and aspartame and flavor pass through #30 sieve;
3. Prelubrication of above materials in blender for 15 minutes with 10 RPM;
4. Sifting the PRUV pass through #40 sieve;
5. Lubrication of above materials in blender for 5 minutes with 10 RPM; and
6. Direct compression with 22 mm×10 mm oval shaped punch.

### Example 4: Omega-3 Fatty, Co-Enzyme Q10, Taurine with Multivitamin & Multimineral Effervescent Tablet

| | | Concentration | |
|---|---|---|---|
| No. | Ingredients (Specific form) | (per tablet) | |
| 1. | Omega 3 | 800 | mg |
| 2. | Co-enzyme (CoQ10) | 100 | mg |
| 3. | Taurine (2-aminoethanesulfonic acid) | 400 | mg |
| 4. | Vitamin B6 (Pyridoxine hydrochloride) | 2.4 | mg |
| 5. | Vitamin B9 (Folic acid) | 300 | mcg |
| 6. | Vitamin B12 (Cyanocobalamin) | 2.2 | mcg |
| 7. | Vitamin D3 (cholecalciferol) | 15 | mcg |
| 8. | Vitamin E (tocopherol) | 10 | mg |
| 9. | Zinc | 17 | mg |
| 10. | Magnesium | 300 | mg |
| 11. | Manganese | 2 | mg |
| 12. | Sodium Selenite | 40 | mcg |

Inactive Ingredients

| 13. | Mannitol | 241 | mg |
|---|---|---|---|
| 14. | Sorbitol | 454.8 | mg |
| 15. | Sodium Bicarbonate | 280 | mg |
| 16. | Tartaric Acid | 10 | mg |
| 17. | Orange Flavour Permaseal | q.s | |
| 18. | Aspartame | 30 | mg |
| 19. | PRUV (Sodium Stearyl Fumarate) | 18.5 | mg |

| Total Tablet weight | Around |
|---|---|
| | 2800 mg |
| | q.s.: Quantity sufficient |

### Manufacturing Process:

1. Sifting the all Active of serial number 1-12 with diluents by cosifting with #30 sieve;
2. Sorbitol, Sodium Bicarbonate, Tartaric Acid and aspartame and flavor pass through #30 sieve;
3. Prelubrication of above materials in blender for 15 minutes with 10 RPM;
4. Sifting the PRUV pass through #40 sieve;
5. Lubrication of above materials in blender for 5 minutes with 10 RPM; and
6. Direct compression with 22 mm×10 mm oval shaped punch.

### Example 5: Omega-3 Fatty, Co-Enzyme Q10, Taurine with Multivitamin & Multi-Mineral Chewable Tablet

| | Ingredients | Concentration | |
|---|---|---|---|
| No. | (Specific form) | (per tablet) | |
| 1. | Omega 3 | 800 | mg |
| 2. | Co-enzyme (CoQ10) | 100 | mg |
| 3. | Taurine (2-aminoethanesulfonic acid) | 400 | mg |
| 4. | Vitamin B6 (Pyridoxine hydrochloride) | 2.4 | mg |
| 5. | Vitamin B9 (Folic acid) | 300 | mcg |
| 6. | Vitamin B12 (Cyanocobalamin) | 2.2 | mcg |
| 7. | Vitamin D3 (cholecalciferol) | 15 | mcg |
| 8. | Vitamin E (tocopherol) | 10 | mg |
| 9. | Zinc | 17 | mg |
| 10. | Magnesium | 300 | mg |
| 11. | Manganese | 2 | mg |
| 12. | Sodium Selenite | 40 | mcg |
| 13. | Inactive Ingredients | | |
| 14. | Sucrose | 450 | mg |
| 15. | Methyl Hydroxybenzoate | 0.01 | mg |
| 16. | Propyl Hydroxybenzoate | 0.01 | mg |
| 17. | Aspartame | 20 | mg |
| 18. | Acacia Powder | 1 | mg |
| 19. | Citric Acid Monohydrate | 1.0 | mg |
| 20. | Magnesium Stearate | 1.5 | mg |
| 21. | Colloidal Silicone Dioxide | 0.2 | mg |

| | | |
|---|---|---|
| 22. | Flavour Fruit DC/DCS 130 PH (Dry Mix) | q.s |
| 23. | Pearlitol ® 200 SD Mannitol | q.s. |
| 24. | Purified Water | q.s |

| Total Tablet weight | Around |
|---|---|
| | 1800 mg |

### Manufacturing Process:

1. Sifting the all Active of serial number 1-12 and sucrose though with #30 sieve;
2. Dry mixing of above materials in RMG for 10 minutes with 12 RPM;
3. Binder preparation with Acacia powder in sufficient qty of water and addition of binder in above dry mixed materials;
4. Sifting the though with #10 sieve. And drying at 35° C. to achieve the desired LOD is below 3%;
5. Shifting of dry granules through #24 sieve;
6. Prelubrication of above dry granules along with Methyl Hydroxybenzoate, Propyl Hydroxybenzoate, Pearlitol® 200 SD Mannitol, Colloidal Silicone Dioxide, Citric Acid Monohydrate, Flavour Fruit and Aspartame in blender for 10 min with RPM 12;
7. Sifting of Magnesium Stearate with through #60 sieve;
8. Blending of above materials in blender for 5 minutes with suitable 10 RPM; and
9. Lubricated blend for compression with 18 mm×9 mm oval shaped punch

1. An oral pharmaceutical or nutraceutical composition comprising:
a) One or more vitamin;
b) One or more mineral;
c) One or more coenzyme;
d) One or more amino acid;
e) One or more omega-3 fatty acid; and
f) One or more pharmaceutically acceptable excipient.

2. The composition as claimed in claim 1, comprising:
a) 0.001 mg to 50 mg of One or more vitamin;
b) 0.001 mg to 500 mg of One or more mineral;
c) 1 mg to 200 mg of One or more coenzyme;
d) 1 mg to 800 mg of One or more amino acid;
e) 1 mg to 1600 mg of One or more omega-3 fatty acid; and
f) One or more suitable pharmaceutically acceptable excipient.

3. The composition as claimed in claim 1, wherein the vitamin is selected from a group consisting of Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K and combinations thereof.

4. The composition as claimed in claim 1, wherein the mineral is selected from a group consisting of calcium, phosphorus, potassium, sodium, magnesium, sulfur, iron, chlorine, cobalt, copper, zinc, manganese, molybdenum, iodine, selenium, chromium and combinations thereof.

5. The composition as claimed in claim 1, wherein coenzyme is coenzyme Q10 (ubiquinone).

6. The composition as claimed in claim 1, wherein the amino acid is selected from a group consisting of taurine (tauric acid), leucine, arginine, glutamate, glycine and combinations thereof.

7. The composition as claimed in claim 1, wherein the omega-3 fatty acid is selected from a group consisting of docosahexaenoic acid (DHA), Alpha-linolenic acid (ALA) and eicosapentaenoic acid (EPA), and combinations thereof.

8. The composition as claimed in claim 1, wherein the pharmaceutically acceptable excipient is selected from a group consisting of diluents, binders, disintegrants, lubricants, glidants, filler, preservative, antioxidants, taste masking agents, chelating agent, thickening agent, flavoring agents, coating agents, coloring agents, solvent(s), cosolvent(s), vehicle, antiadherents, preservatives, sorbents, sweeteners, release-controlling polymers, stabilizing agent, acidic agent, basic agent, buffering agent, effervescent agent, pH regulating agent and combinations thereof.

9. The composition as claimed in claim 1, wherein the composition comprises tablet, effervescent tablet, chewable tablet, modified release tablet, controlled release tablet, delayed release tablet, capsule, granules, pellets, beads or sachet, oral syrup, liquid oral solution, solution, suspension, or emulsion dosage form.

10. A process for preparation of a novel oral pharmaceutical or nutraceutical composition, comprising:
a) Dissolving multivitamins, minerals, coenzyme, amino acid and omega-3 fatty acids into a solvent or a vehicle to obtain a solution;
b) Adding and dissolving one or more pharmaceutically acceptable excipient into the solution obtained in step (a);
c) Filtering the solution obtained in step (b) through suitable filter; and
d) Filling the filtered solution obtained in step (c) into a bottle or a container.

11. A process for preparation of an oral pharmaceutical or nutraceutical composition, comprising:
a) Sifting multivitamins, minerals, coenzyme, amino acid and omega-3 fatty acids through a sieve;
b) Sifting one or more pharmaceutically acceptable excipient through a sieve;
c) pre-lubricating the sifted material obtained in step (a) and step (b), and blending with a blender to obtain a blended material;
d) Lubricating above blended material obtained in step (c) and continued blending it for 5 to 10 minutes; and
e) Compressing the lubricated blend of step (d) into a dosage form.

12. A process for preparation of an oral pharmaceutical or nutraceutical composition, comprising:
a) Sifting multivitamins, minerals, coenzyme, amino acid and omega-3 fatty acids through a sieve;
b) Dry mixing of the components of step (a);
c) Preparing a binder solution;
d) Granulating the mixture obtained in step (b) using the binder solution obtained in step (c);
e) Drying the granules obtained in step (d);
f) Sifting, blending and lubricating the dried granules of step (e); and
g) Compressing the granules of step (f) into a dosage form.

13. A method of treating a patient in need of support or prophylactic treatment for epilepsy or seizures, comprising administering an amount of the composition of claim effective to prevent or treat epilepsy or seizures in all age groups of patients to the patient.
